(19) United States
(12) Patent Application Publication
(10) Pub. No.: US 2014/0206950 A1
(43) Pub. Date: Jul. 24, 2014

(54) WARD CLOUD SYSTEM

(71) Applicant: OSTAR MEDITECH CORP., New Taipei City (TW)

(72) Inventor: Kuo-Yuan CHANG, New Taipei City (TW)

(73) Assignee: OSTAR MEDITECH CORP., New Taipei Ctiy (TW)

(21) Appl. No.: 14/159,439

(22) Filed: Jan. 21, 2014

(30) Foreign Application Priority Data
Jan. 18, 2013 (TW) 102102038

(51) Int. Cl. A61B 5/00

(52) U.S. Cl. CPC A61B 5/0022; A61B 5/742
USPC 600/301

(57) ABSTRACT

A ward cloud system comprises an intelligent cloud database and an intelligent electronic vital-sign monitoring device. The intelligent cloud database receives first vital-sign information detected by the intelligent electronic vital-sign monitoring device and second vital-sign information manually input by users through an intelligent transmission unit, integrates received information and undertakes far-end clock synchronization of the intelligent cloud database and intelligent electronic vital-sign monitoring device. The ward cloud system further comprises a far-end calibration unit using the intelligent cloud database to examine and calibrate accuracy of the intelligent electronic vital-sign monitoring device through the intelligent transmission unit from a far end. The present invention adds to an ordinary electronic vital-sign monitoring system the following functions: a manual vital-sign value input function, a far-end automatic clock synchronization function, a far-end calibration function and an intelligent operation function and thus can avoid the serious consequences caused by measurement inaccuracy or clock non-synchronicity.

### BACKGROUND OF THE INVENTION

1. Field of the Invention

The present invention relates to a ward cloud system, particularly to a multifunctional physiological monitor system with an intelligent cloud database, such as a blood pressure monitor, a blood oxygen monitor, an electrocardiograph monitor, a body temperature monitor, a respiratory monitor, a glucose monitor, or a patient monitor.

2. Description of the Related Art

Medical personnel need to measure and record basic vital signs of inpatients, including heart rate, respiratory rate, blood pressure, blood oxygen, body temperature, electrocardiograph, urine volume, stool volume, pain score, food intake volume, and fluid infusion volume. In the past, medical personnel record vital signs manually. The modern hospitals normally adopt electronic anamneses and use electronic physiological monitoring systems to automatically transmit vital-sign data of patients. However, most of the vital signs still need to be measured, transcribed and then input into computers by medical personnel manually, which is inefficient and laborious. Further, manual transcription and inputting is likely to have errors, especially while the medical personnel are busy at work or while they are handing over. Furthermore, errors are also likely to occur between the clocks of medical instruments and the clock of the hospital. The erroneous information would result in errors of administration and therapy. Therefore, the present invention provides a manual vital-sign data input function, which not only saves medical personnel the time of transcription but also avoids inaccuracy of manual transcription and errors of vital-sign data transference. In addition to small medical electronics measuring basic vital signs (such as blood pressure and heart rate), the patients discharged from hospitals further need sophiscated remote electronic vital-sign monitor systems, which can cooperate with the healthcare system of the hospital to far-end track the states of the discharged patients. While undertaking daily cloud-based vital-sign monitoring of the discharged patients, the caregivers or family members use medical instruments to measure heart rate, blood pressure, blood oxygen, blood glucose, body temperature, and/or electrocardiograph and input the vital-sign data, such as respiratory rate, blood glucose, body temperature, body weight, food intake volume, fluid infusion volume, urine volume, stool volume and/or pain score to the system, whereby the caregivers can record and track their health states daily and transmit the data to the healthcare system.

Most of the commercially-available physiological measurement devices or physiological monitors can measure, calculate and display. However, they lack functions of cloud operation, clock synchronization, and self-calibration. Therefore, they need self-calibrating and clock-synchronizing after they have been used for a period of time.

For example, the physicians administrate medication according to the results of blood pressure measurements, and the inpatients take blood pressure medicine on time. Errors of the instrument clocks may cause the physicians to make wrong medicine administration or cause the patients to take medicine in wrong timing Clock non-synchronicity between instruments and medical personnel may cause confusion of treatment and nursing or even endanger the lives of patients. For example, the time of physician prescription is earlier than the time of blood pressure measurement, and the patients take antihypertensive medicine while they are in a low-blood pressure state.

Some regions have great temperature difference. For example, in the northeast of China, the temperature is −27° C. outdoors and 18° C. indoors. The circuit board of an electronic hemadynamometer is distorted outdoors and distorted once again after it has been taken back to the house and restored to the indoor temperature. Thus, the electronic hemadynamometer may have measurement errors. The circuit board of an electronic hemadynamometer may also be distorted after it has been stored for a long time. Then, the electronic hemadynamometer may also have measurement errors. In a high altitude, the calibration values of an electronic hemadynamometer may become inaccurate. Hence, an electronic hemadynamometer cannot undertake a precise measurement in a high altitude area. In the abovementioned cases, the electronic hemadynamometers need sending to the manufacturer for recalibration, which is money-, time- and labor-consuming and besets the users who need to monitor their blood pressure every day.

As the ordinary electronic vital-sign measurement devices are easy to carry and easy to operate, they are very popular in hospitals, clinics, elderly nursing centers, and nursing homes. However, medical personnel can neither manually input vital-sign data into the ordinary electronic vital-sign measurement devices nor link the ordinary electronic vital-sign measurement devices to cloud databases. Therefore, the ordinary electronic vital-sign measurement devices cannot automatically monitor the patients needing emergent nursing or the elders needing special nursing.

The medical personnel of hospitals will work more easily and efficiently if they can further manually input vital-sign data into ordinary electronic vital-sign measurement devices (such as electronic devices for measuring pulse, respiration, blood pressure, blood oxygen, blood glucose, body temperature, body weight and electrocardiograph) in realtime and then transmit the vital-sign data to cloud databases while measuring vital signs of patients.

The telecare centers, which have adopted ordinary electronic vital-sign measurement devices to measure vital signs, such as pulse, respiration, blood pressure, blood oxygen, blood glucose, body temperature and electrocardiograph, will be able to monitor and administrate the vital-sign data of patients, elders and testees in real time if the nursing personnel can further manually input vital-sign data into the ordinary electronic vital-sign measurement devices and then transmit the vital-sign data to cloud databases.

Accordingly, the Inventors develop a ward cloud system, which enables hospitals, clinics, nursing centers, physicians, nursing personnel, patients and elders to monitor vital signs and heart state in realtime and transmit the measured vital-sign data to a cloud database, whereby the users can provide or acquire more assistance.

### SUMMARY OF THE INVENTION

One objective of the present invention is to provide a ward cloud system, which can detect vital signs, enables the users to input other vital-sign data manually to the ward cloud system, and can transmit the detected and manually-input vital-sign data to an intelligent cloud database, and which can integrate the data, automatically evaluate the data intelligently, and inform an abnormal state, and which can undertake clock synchronization and far-end calibration. Therefore, the present invention can provide appropriate assistance for hospitals, clinics, nursing centers, physicians, nursing personnel, patients, elders and ordinary users. In the present invention, the database is HIS (Hospital Information System), NIS (Nursing Information System), an HL7 (Health Level Seven) database, or an ordinary intelligent cloud database. If the database is an ordinary intelligent cloud database, the database can link to at least one of HIS, NIS, HL7, an emergency system, an intensive care system, or a clinic system, and can exchange information with them. The far-end clock synchronization function of the present invention can calibrate the clock of the system automatically, wherein the present invention uses an intelligent cloud database to receive the vital-sign data obtained by an intelligent electronic monitoring device through an intelligent transmission unit and integrates the vital-sign data, and wherein the present invention undertake clock synchronization between the intelligent cloud database and the intelligent electronic vital-sign monitoring device from a far end. Further, the present invention can examine and calibrate the accuracy of the intelligent electronic vital-sign monitoring device from a far end and exempts the device from being sent back to the manufacturer for calibration by special instruments or extra manual set-up. Therefore, the present invention can undertake clock synchronization, examination and calibration from a far end without extra expense of money and labor. Another objective of the present invention is to provide a ward cloud system, which can detect vital signs, enables the users to input other vital-sign data manually to the ward cloud system, and can transmit the detected and manually-input vital-sign data to an intelligent cloud database, and which possesses a far-end healthcare function and an intelligent cloud data processing function, wherein the intelligent cloud data processing function can automatically evaluate and deal with various cases and access the cloud database, whereby the present invention can provide appropriate assistance for hospitals, clinics, nursing centers, physicians, nursing personnel, patients, elders and ordinary users.

To achieve the abovementioned objectives, the present invention proposes a ward cloud system, which comprises an intelligent electronic vital-sign monitoring device and an intelligent cloud database. The intelligent cloud database receives vital-sign information, integrates the vital-sign information, and undertakes far-end clock synchronization and intelligent computation. The intelligent electronic vital-sign monitoring device includes a detection unit detecting first vital-sign information; a vital-sign recording unit enabling the user to manually input second vital-sign information; a calculating/processing unit connected with the detection unit and the vital-sign recording unit, calculating the first vital-sign information or the second vital-sign information, or processing clock synchronization information; an identity verification unit connected with the calculating/processing unit and recording identity information of users, medical personnel, and patients; an intelligent transmission unit connected with the calculating/processing unit, transmitting the first vital-sign information, the second vital-sign information, or the identity information to the intelligent cloud database in a wired or wireless way, receiving the clock synchronization information from the intelligent cloud database for system clock synchronization; a display unit connected with the calculating/processing unit and presenting the first vital-sign information or the second vital-sign information; and a power supply unit connected with the detection unit, the vital-sign recording unit, the calculating/processing unit, the identity verification unit, the intelligent transmission unit and the display unit for charging or powering them.

In one embodiment, the first vital-sign information is information of at least one of pulse, blood pressure, blood oxygen, blood glucose, body temperature, body weight, and electrocardiograph.

In one embodiment, the second vital-sign information is information of at least one of respiration rate, blood glucose, body temperature, body weight, food intake volume, fluid infusion volume, urine volume, stool volume and pain score.

In one embodiment, the ward cloud system further comprises a cloud calibration unit using the intelligent cloud database to far-end examine and calibrate the accuracy of the intelligent electronic vital-sign monitoring device through the intelligent transmission unit.

In one embodiment, the ward cloud system controls the intelligent cloud database to undertake clock synchronization of the intelligent electronic vital-sign monitoring device at system initiation, after vital-sign measurement, or at an arbitrary timing. In one embodiment, the intelligent electronic vital-sign monitoring device transmits a clock synchronization request to the intelligent cloud database at system initiation, after vital-sign measurement, or at an arbitrary timing.

In one embodiment, the vital-sign recording unit is realized by a function button or a knob, which can be pressed or rotated to adjust the value so as to manually input one or more types of the second vital-sign information. In one embodiment, the vital-sign recording unit is realized by more than one function button or more than one knob, which can be pressed or rotated to adjust the value so as to manually input one or more types of the second vital-sign information. In one embodiment, the vital-sign recording unit is realized by a touchscreen. The user clicks on the option items of the touchscreen to adjust the value so as to manually input one or more types of the second vital-sign information.

In one embodiment, the intelligent transmission unit is a wired transmission interface, such as a USB port or an RS232 port. In one embodiment, the intelligent transmission unit is a wireless transmission interface, such as a 2.4G or WiFi transmission interface. In one embodiment, the ward cloud system is connected with an ID scanner, such as a bar-code scanner, a card reader, an RFID (radio frequency identification) reader, or an NFC (Near Field Communication) device.

In one embodiment, the intelligent electronic vital-sign monitoring device or the intelligent cloud database can automatically detect a warning value of the first vital-sign information or the second vital-sign information according to a preset standard. The ward cloud system can automatically detect whether one or more pieces of first vital-sign information or second vital-sign information are transmitted completely and can automatically transmit the information once again if the information transmission was interrupted. The display unit can present one or more transmission states of the first vital-sign information or the second vital-sign information, such as transmission completion, transmission failure, transmission standby, link completion, and link failure, and present the states with different text or symbols. After having received the first vital-sign information or the second vital-sign information, the intelligent cloud database automatically undertakes statistics and computation of the first vital-sign information or the second vital-sign information. The ward cloud system automatically informs the related persons of the occurrence of the first vital-sign information, the second vital-sign information, or the warning value in a wired or wireless way, such as email, SMS, or MMS. While transmitting the first vital-sign information or the second vital-sign information via the intelligent transmission unit, the calculating/processing unit automatically encrypts the information lest the information be exposed. After having received the first vital-sign information or the second vital-sign information, the intelligent cloud database automatically encrypts the information lest the information be exposed. The intelligent electronic vital-sign monitoring device can be used by the patient at the hospital or home. The intelligent cloud database can receive the first vital-sign information or the second vital-sign information of inpatients, outpatients or discharged patients. In the present invention, the intelligent cloud database is HIS (Hospital Information System), NIS (Nursing Information System), an HL7 (Health Level Seven) database, or an ordinary intelligent cloud database. If the intelligent cloud database is an ordinary intelligent cloud database, the intelligent cloud database can link to at least one of HIS, NIS, HL7, an emergency system, an intensive care system, or a clinic system, and can exchange information with them.

In one embodiment, the information of the heart is information of an electrocardiograph or information of a heart spectrum. The heart spectrum is obtained via measuring the pulse signals of the heart and transforming the pulse signals into a spectrum diagram with an FFT (Fast Fourier Transform) algorithm. The spectrum diagram normally has 3-5 main frequency waveforms. The peak of the first main frequency waveform is the frequency of heart beats. If there are also several intricate frequency waveforms appearing in the spectrum, it indicates that the heart beats irregularly. In such a case, the heart is regarded as in an abnormal state. Therefore, the heart spectrum can be used to evaluate the function of the heart. The related technology has been disclosed in a Taiwan patent No. I280119 proposed by the Inventors and will not repeat herein.

### BRIEF DESCRIPTION OF THE DRAWINGS

FIG. 1 is a block diagram schematically showing a ward cloud system according to one embodiment of the present invention; and

FIG. 2 is a flowchart of the measurement process of a ward cloud system according to one embodiment of the present invention.

### DETAILED DESCRIPTION OF THE INVENTION

Below, the present invention is described in detail with embodiments. However, it should be understood that these embodiments are only to exemplify the present invention but not to limit the scope of the present invention.

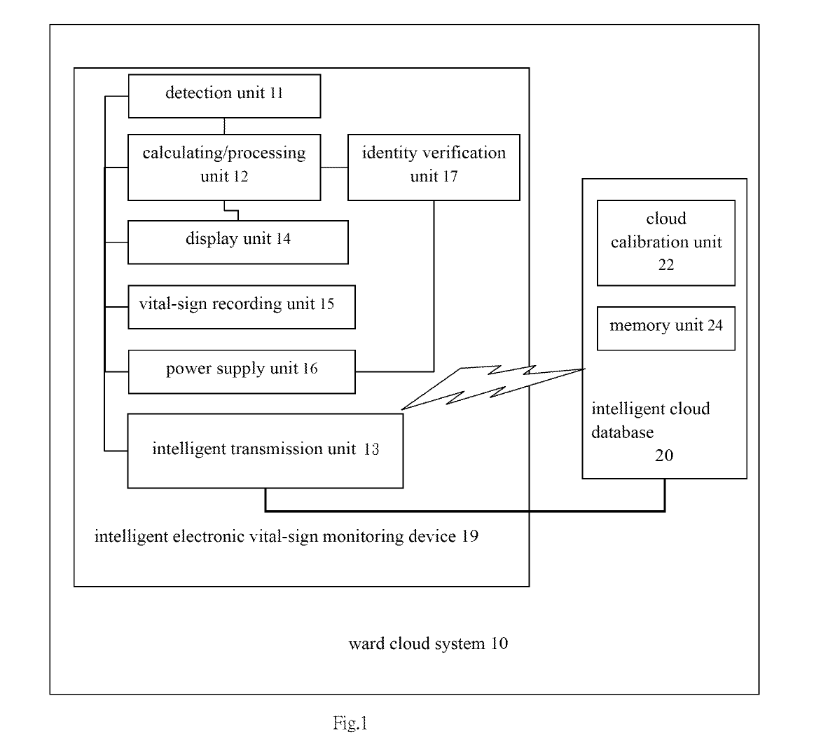

Refer to FIG. 1 a block diagram schematically showing a ward cloud system according to one embodiment of the present invention. The ward cloud system 10 of the present invention comprises an intelligent electronic vital-sign monitoring device 19 and an intelligent cloud database 20. The intelligent cloud database 20 receives vital-sign information, integrates the vital-sign information, and undertakes far-end clock synchronization and intelligent computation. The intelligent electronic vital-sign monitoring device 10 includes a detection unit 11 detecting first vital-sign information; a vital-sign recording unit 15 enabling the user to manually input second vital-sign information; a calculating/processing unit 12 connected with the detection unit 11 and the vital-sign recording unit 15, calculating the first vital-sign information and the second vital-sign information, and processing clock synchronization information; an identity verification unit 17 connected with the calculating/processing unit 12 and recording identity information of users, medical personnel, and patients; an intelligent transmission unit 13 connected with the calculating/processing unit 12 and transmitting the first vital-sign information, the second vital-sign information, and the identity information to the intelligent cloud database 20 in a wired or wireless way; a display unit 14 connected with the calculating/processing unit 12 and presenting the first vital-sign information and the second vital-sign information; and a power supply unit 16 connected with the detection unit 11, the vital-sign recording unit 15, the calculating/processing unit 12, the identity verification unit 17, the intelligent transmission unit 13 and the display unit 14 for charging or powering them. The intelligent cloud database 20 provides clock information for the intelligent electronic vital-sign monitoring device 19. The calculating/processing unit 12 compares the clock information with the clock of the intelligent electronic vital-sign monitoring device 19 and undertakes clock synchronization of the intelligent electronic vital-sign monitoring device 19 and the intelligent cloud database 20.

In one embodiment, at initiation of the ward cloud system 10, after the intelligent electronic vital-sign monitoring device 19 has detected the first vital-sign information, or at an arbitrary timing, the intelligent electronic vital-sign monitoring device 19 transmits a clock synchronization request to the intelligent cloud database 20 beforehand, then the intelligent cloud database 20 supplies clock information to the intelligent electronic vital-sign monitoring device 19 for clock synchronization.

In one embodiment, at initiation of the ward cloud system 10, after the intelligent electronic vital-sign monitoring device 19 has detected the first vital-sign information, or at an arbitrary timing, the intelligent cloud database 20 transmits a clock synchronization request to the intelligent electronic vital-sign monitoring device 19, and the intelligent electronic vital-sign monitoring device 19 transmits an acknowledge message to the intelligent cloud database 20. Then, the intelligent cloud database 20 supplies clock information to the intelligent electronic vital-sign monitoring device 19 for clock synchronization.

The clock of the intelligent electronic vital-sign monitoring device 19 may become inaccurate because of long-term usage or errors made by manual setting. Errors of the instrument clocks may cause the physicians to make wrong medicine administration or cause the patients to take medicine in wrong timing. For example, the patients take antihypertensive medicine while they are in a low-blood pressure state. Therefore, non-synchronicity of clocks may seriously impair the nursing quality or even endanger the patient. Thus, the present invention is designed to unify the clocks of the ward cloud system and undertake far-end clock synchronization to overcome the abovementioned problems.

The ward cloud system 10 further comprises a cloud calibration unit 22, which can examine and calibrate the accuracy of the intelligent electronic vital-sign monitoring device 19 via the intelligent transmission unit 13 from a far end. While the intelligent electronic vital-sign monitoring device 19 is turned on, the intelligent cloud database 20 obtains a vital-sign measurement value of the intelligent electronic vital-sign monitoring device 19 through the intelligent transmission unit 13. The cloud calibration unit 22 compares the vital-sign measurement value with a calibration value preset by the manufacturer to examine and calibrate the accuracy of the intelligent electronic vital-sign monitoring device 19. In one embodiment, the intelligent electronic vital-sign monitoring device 19 is an electronic hemadynamometer, and the vital-sign measurement value is a blood pressure value. While the intelligent electronic vital-sign monitoring device 19 is turned on, the intelligent cloud database 20 obtains a pressure value from the intelligent electronic vital-sign monitoring device 19 through the intelligent transmission unit 13. The cloud calibration unit 22 compares the pressure value with a pressure calibration value provided by the manufacturer (such as the position of 0 mmHg). If the pressure value of the intelligent electronic vital-sign monitoring device 19 is deviated from 0 mmHg, the intelligent cloud database 20 calibrates the intelligent electronic vital-sign monitoring device 19 to offset the deviation.

The ward cloud system 10 further comprises a memory unit 24 recording the calibration values provided the manufacturer and the offset values for device calibration. While the ward cloud system 10 is turned on, or at specified timings, the intelligent cloud database 20 compares the calibration value provided by the manufacturer with the measurement value of the intelligent electronic vital-sign monitoring device 19 and calibrates the intelligent electronic vital-sign monitoring device 19. If the measurement values of the intelligent electronic vital-sign monitoring device 19 are within the tolerance, the display unit 14 automatically indicates that the intelligent electronic vital-sign monitoring device 19 is normal. If the measurement value of the intelligent electronic vital-sign monitoring device 19 exceeds the tolerance, the ward cloud system 10 calibrates the intelligent electronic vital-sign monitoring device 19 and stores he offset values for device calibration in the memory unit 24.

In one embodiment, the first vital-sign information is information of at least one of pulse, blood pressure, blood oxygen, blood glucose, body temperature, and body weight, and electrocardiograph.

In one embodiment, the second vital-sign information is information of at least one of respiration rate, blood glucose, body temperature, body weight, food intake volume, fluid infusion volume, urine volume, stool volume and pain score.

In one embodiment, the vital-sign recording unit 15 is realized by a function button or a knob, which can be pressed or rotated to adjust the value so as to manually input one or more types of the second vital-sign information. In one embodiment, the vital-sign recording unit 15 is realized by more than one function button or more than one knob, which can be pressed or rotated to adjust the value so as to manually input one or more types of the second vital-sign information. In one embodiment, the vital-sign recording unit 15 is realized by a touchscreen. The user clicks on the option items of the touchscreen to adjust the value so as to manually input one or more types of the second vital-sign information.

In one embodiment, the intelligent transmission unit 13 is a wired transmission interface, such as a USB port or an RS232 port. In one embodiment, the transmission unit is a wireless transmission interface, such as a 2.4G or WiFi transmission interface. In one embodiment, the ward cloud system 10 is connected with an ID scanner, such as a bar-code scanner, a card reader, an RFID (radio frequency identification) reader, or an NFC (Near Field Communication) device.

In one embodiment, the intelligent electronic vital-sign monitoring device 10 or the intelligent cloud database 20 can automatically detect a warning value of the first vital-sign information or the second vital-sign information according to a preset standard. The ward cloud system 10 can automatically detect whether one or more pieces of first vital-sign information or second vital-sign information are transmitted completely and can automatically transmit the information once again if the information transmission was interrupted. The display unit 14 can present one or more transmission states of the first vital-sign information or the second vital-sign information, such as transmission completion, transmission failure, transmission standby, link completion, and link failure, and present the states with different text or symbols. After having received the first vital-sign information or the second vital-sign information, the intelligent cloud database 20 automatically undertakes statistics and computation of the first vital-sign information or the second vital-sign information. The ward cloud system 10 automatically informs the related persons of the occurrence of the first vital-sign information, the second vital-sign information, or the warning value in a wired or wireless way, such as email, SMS, or MMS. While transmitting the first vital-sign information or the second vital-sign information via the intelligent transmission unit 13, the calculating/processing unit 12 automatically encrypts the information lest the information be exposed. After having received the first vital-sign information or the second vital-sign information, the intelligent cloud database 20 automatically encrypts the information lest the information be exposed. The intelligent electronic vital-sign monitoring device 19 can be used by the patient at the hospital or home. The intelligent cloud database 20 can receive the first vital-sign information or the second vital-sign information of inpatients, outpatients or discharged patients. In the present invention, the intelligent cloud database 20 is HIS (Hospital Information System), NIS (Nursing Information System), an HL7 (Health Level Seven) database, or an ordinary intelligent cloud database. If the intelligent cloud database 20 is an ordinary intelligent cloud database, the intelligent cloud database 20 can link to at least one of HIS, NIS, HL7, an emergency system, an intensive care system, or a clinic system, and can exchange information with them. Therefore, the ward cloud system 10 of the present invention can link to at least one of HIS, NIS, HL7, an emergency system, an intensive care system, or a clinic system, and can exchange information with them. Thus, the ward cloud system 10 of the present invention can smooth exchange or mediate information.

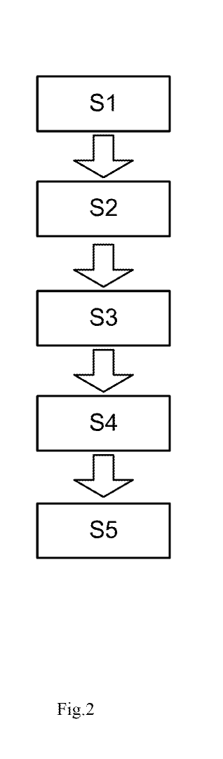

Refer to FIG. 2 for a flowchart of the measurement process of a ward cloud system according to one embodiment of the present invention. In this embodiment, the ward cloud system 10 is a blood pressure and heart information measurement system. In Step S1, a detection unit 11 measures the pulse of a patient or testee. In Step S2, a calculating/processing unit 12 calculates the pulse information and obtains blood pressure information. In Step S3, the user uses a vital-sign recording unit 15 to input the respiratory rate of the patient or testee. For example, one or more function buttons, one or more knobs, or a touchscreen can be used to input one or more pieces of respiratory rate information. In Step S4, the calculating/processing unit 12 transmits the pulse information, the blood pressure information and personal information obtained with an ID scanner to an intelligent cloud database 20 via an intelligent transmission unit 13. In Step S5, the intelligent cloud database 20 automatically analyzes and integrates the vital-sign information and personal information. The intelligent cloud database 20 is HIS (Hospital Information System), NIS (Nursing Information System), an HL7 (Health Level Seven) database, or an ordinary intelligent cloud database. If the intelligent cloud database 20 is an ordinary intelligent cloud database, the intelligent cloud database 20 can link to at least one of HIS, NIS, HL7, an emergency system, an intensive care system, or a clinic system, and can exchange information with them. After receiving vital-sign data, the intelligent cloud database 20 processes and files the data immediately, whereby the testees, the family members, the physicians, and the nursing personnel can browse the data through the network. The pulse information, blood pressure information and respiratory rate information can also be presented on a display unit 14, such as an LCD display unit or an LED display device. A power supply unit 16 provides power for the system. In some embodiments, the power supply unit 16 is an alkaline battery assembly, a rechargeable battery assembly, a capacitor, or a power supply device.

In the present invention, the ward cloud system 10 is a unifunctional vital-sign measurement system or a multifunction patient monitoring system.

The present invention adds to an ordinary electronic vital-sign monitoring system the following functions: a second vital-sign value input function, a far-end clock synchronization function, a far-end examination and calibration function, and a transmission function. An ordinary electronic vital-sign monitoring system using the present invention is able to provide instant vital-sign information for the patients, elders, or testees, who need routine healthcare or immediate medical assistance. Further, the intelligent cloud database 20 enables the testees, family members, physicians, and nursing personnel to track vital-sign information in a wired or wireless way (such as via the Internet). Therefore, the present invention can realize far-end healthcare service.

The present invention proposes a ward cloud system 10, which can fast and precisely detect and record vital-sign data of patients, elders or testees who need routine healthcare or immediate medical assistance and can instantly provide the vital-sign data for family members, telecare centers, personnel of nursing stations or physicians. Suppose that an abnormal state appears in a patient whose vital signs are being continually monitored by the ward cloud system 10, such as a blood pressure monitor system, a blood oxygen monitor system, a blood glucose monitor system, a patient monitor system or an electrocardiograph monitor system. In such a case, the related personnel should enquire the patient about his feeling as soon as possible and instantly input the abnormal vital-sign values into the system manually. The related personnel should send the patient to a large-scale hospital or medical center for further examination if necessary.

In conclusion, the present invention proposes a ward cloud system 10, wherein the original measurement function, an information transmission function, a manual vital-sign data input function, a far-end clock synchronization function, a far-end examination and calibration function, and a far-end healthcare function are integrated in a system to provide appropriate assistance for patients, elders and testees.

Although the present invention has been explained in relation to its preferred embodiment, it is to be understood that other modifications and variation can be made without departing the spirit and scope of the invention as hereafter claimed.

What is claimed is:

1. A ward cloud system comprising
an intelligent electronic vital-sign monitoring device including
a detection unit detecting first vital-sign information of a patient;
a vital-sign recording unit used to manually input second vital-sign information;
a calculating/processing unit connected with said detection unit and said vital-sign recording unit, calculating at least one of said first vital-sign information and said second vital-sign information;
an identity verification unit connected with said calculating/processing unit and recording at least one piece of identity information;
an intelligent transmission unit connected with said calculating/processing unit and transmitting said first vital-sign information, said second vital-sign information, and said identity information in a wired or wireless way;
a display unit connected with said calculating/processing unit and presenting at least one of said first vital-sign information, said second vital-sign information, and said identity information; and
a power supply unit connected with said detection unit, said vital-sign recording unit, said calculating/processing unit, said identity verification unit, said intelligent transmission unit, and said display unit for charging or powering them; and
an intelligent cloud database receiving and integrating said first vital-sign information, said second vital-sign information and said identity information transmitted by said intelligent transmission unit, and providing clock information for said intelligent electronic vital-sign monitoring device, wherein said calculating/processing unit compares said clock information with a clock thereof and undertakes clock synchronization of said intelligent electronic vital-sign monitoring device and said intelligent cloud database.

2. The ward cloud system according to claim 1, wherein after said ward cloud system is turned on, said intelligent electronic vital-sign monitoring device transmits a clock synchronization request to said intelligent cloud database beforehand, and said intelligent cloud database supplies said clock information to said intelligent electronic vital-sign monitoring device for clock synchronization.

3. The ward cloud system according to claim 1, wherein after said intelligent electronic vital-sign monitoring device has detected said first vital-sign information, said intelligent electronic vital-sign monitoring device transmits a clock synchronization request to said intelligent cloud database beforehand, and said intelligent cloud database supplies said clock information to said intelligent electronic vital-sign monitoring device for clock synchronization.

4. The ward cloud system according to claim 1, wherein after said ward cloud system is turned on, said intelligent cloud database transmits a clock synchronization request to said intelligent electronic vital-sign monitoring device; next, said intelligent electronic vital-sign monitoring device returns an acknowledge message to said intelligent cloud database; next, said intelligent cloud database supplies said clock information to said intelligent electronic vital-sign monitoring device for clock synchronization.

5. The ward cloud system according to claim 1, wherein after said intelligent electronic vital-sign monitoring device has detected said first vital-sign information, said intelligent cloud database transmits a clock synchronization request to said intelligent electronic vital-sign monitoring device; next, said intelligent electronic vital-sign monitoring device returns an acknowledge message to said intelligent cloud database; next, said intelligent cloud database supplies said clock information to said intelligent electronic vital-sign monitoring device for clock synchronization.

6. The ward cloud system according to claim 1 further comprising a cloud calibration unit, wherein after said intelligent electronic vital-sign monitoring device is turned, or at a specified time point, said intelligent cloud database obtains a vital-sign measurement value from said intelligent electronic vital-sign monitoring device through said intelligent transmission unit; next, said cloud calibration unit compares said vital-sign measurement value with a calibration value provided by a manufacturer of said intelligent electronic vital-sign monitoring device to examine and calibrate accuracy of said intelligent electronic vital-sign monitoring device.

7. The ward cloud system according to claim 6 further comprising a memory unit recording said calibration value provided by said manufacturer and an offset value said cloud calibration unit uses to calibrate said accuracy of said intelligent electronic vital-sign monitoring device.

8. The ward cloud system according to claim 1, wherein said identity information includes identity information of users, medical personnel and patients.

9. The ward cloud system according to claim 1, wherein said first vital-sign information is information of at least one of pulse, blood pressure, blood oxygen, blood glucose, body temperature, body weight, and electrocardiograph.

10. The ward cloud system according to claim 1, wherein said second vital-sign information is information of at least one of respiration rate, blood glucose, body temperature, body weight, food intake volume, fluid infusion volume, urine volume, stool volume and pain score.

11. The ward cloud system according to claim 1, wherein said vital-sign recording unit is realized by at least one function button, at least one knob or a touchscreen, which is used to manually input one or more types of the second vital-sign information.

12. The ward cloud system according to claim 1, wherein said intelligent electronic vital-sign monitoring device or said intelligent cloud database automatically detects a warning value of said first vital-sign information or said second vital-sign information according to a preset standard.

13. The ward cloud system according to claim 12, wherein related persons is informed of occurrence of said first vital-sign information, said second vital-sign information, or said warning value in a wired or wireless way inform of email, SMS or MMS.

14. The ward cloud system according to claim 1, wherein said intelligent electronic vital-sign monitoring device or said intelligent cloud database automatically detects whether transmission of one or more pieces of said first vital-sign information or said second vital-sign information is completed, and wherein if said transmission is interrupted, said transmission is undertaken once again.

15. The ward cloud system according to claim 1, wherein said display unit presents one or more transmission states to indicate whether said first vital-sign information or said second vital-sign information has been transmitted to said intelligent cloud database successfully, and wherein said transmission states include transmission completion, transmission failure, transmission standby, link completion and link failure, and wherein said display unit presents said transmission states with different text or symbols.

16. The ward cloud system according to claim 1, wherein while said calculating/processing unit transmits said first vital-sign information or said second vital-sign information via said intelligent transmission unit, said calculating/processing unit automatically encrypts transmitted information.

17. The ward cloud system according to claim 1, wherein after said intelligent cloud database receives said first vital-sign information or said second vital-sign information, said intelligent cloud database automatically encrypts received information.

18. The ward cloud system according to claim 1, wherein said intelligent cloud database is HIS (Hospital Information System), NIS (Nursing Information System), an HL7 (Health Level Seven) database, or an ordinary intelligent cloud database.

19. The ward cloud system according to claim 1, wherein said intelligent cloud database can link to and exchange information with at least one of external systems, and wherein said external systems include HIS (Hospital Information System), NIS (Nursing Information System), an HL7 (Health Level Seven) database, and an ordinary intelligent cloud database.

20. The ward cloud system according to claim 1, wherein said intelligent transmission unit is a 2.4G or WiFi wireless transmission interface.
